(19) United States
(12) Patent Application Publication
(10) Pub. No.: US 2012/0107308 A1
(43) Pub. Date: May 3, 2012

(54) GENE EXPRESSION LEVELS OF EGFR, VEGFR2, AND ERCC1 ASSOCIATED WITH CLINICAL OUTCOMES OF CHEMOTHERAPY

(75) Inventor: Heinz-Josef Lenz, Los Angeles, CA (US)

(73) Assignee: University of Southern California

(21) Appl. No.: 13/265,817

(22) PCT Filed: Apr. 23, 2010
(86) PCT No.: PCT/US10/32251

§ 371 Date: Jan. 11, 2012

Related U.S. Application Data
(60) Provisional application No. 61/172,573, filed on Apr. 24, 2009.

(51) Int. Cl. A61K 39/395; A61K 31/495; C40B 40/06; A61P 35/00; A61K 31/519; A61K 31/282; C12Q 1/68; C40B 30/00

(52) U.S. Cl. 424/133.1; 435/6.12; 435/6.11; 514/256; 506/7; 514/249; 514/492; 506/16

(57) ABSTRACT

The invention provides compositions and methods for identifying a cancer patient suitable for anti-VEGF therapy. After determining if a patient is likely to be successfully treated, the invention also provides methods for treating the patients.

### CROSS-REFERENCE TO RELATED APPLICATION

This application claims the benefit under 35 U.S.C. §119(e) of U.S. Provisional Ser. No. 61/172,573, filed Apr. 24, 2009, the contents of which is incorporated by reference in its entirety.

### STATEMENT AS TO FEDERALLY SPONSORED RESEARCH OR DEVELOPMENT

This invention was made with government support under the National Institutes of Health Grant P30 CA 14078. Accordingly, the U.S. Government has certain rights to the invention.

### FIELD OF THE INVENTION

This invention relates to the filed of pharmacogenomics and specifically to the application of gene expression and genetic polymorphisms to diagnose and treat diseases.

### BACKGROUND OF THE INVENTION

In nature, organisms of the same species usually differ from each other in some aspects, e.g., their appearance. The differences are genetically determined and are referred to as polymorphism. Genetic polymorphism is the occurrence in a population of two or more genetically determined alternative phenotypes due to different alleles. Polymorphism can be observed at the level of the whole individual (phenotype), in variant forms of proteins and blood group substances (biochemical polymorphism), morphological features of chromosomes (chromosomal polymorphism) or at the level of DNA in differences of nucleotides (DNA polymorphism).

Polymorphism also plays a role in determining differences in an individual's response to drugs. Pharmacogenetics and pharmacogenomics are multidisciplinary research efforts to study the relationship between genotype, gene expression profiles, and phenotype, as expressed in variability between individuals in response to or toxicity from drugs. Indeed, it is now known that cancer chemotherapy is limited by the predisposition of specific populations to drug toxicity or poor drug response. For a review of the use of germline polymorphisms in clinical oncology, see Lenz (2004) J. Clin. Oncol. 22(13):2519-2521; Park et al. (2006) Curr. Opin. Pharma. 6(4):337-344; Zhang et al. (2006) Pharma. and Genomics 16(7):475-483 and U.S. Patent Publ. No. 2006/0115827. For a review of pharmacogenetic and pharmacogenomics in therapeutic antibody development for the treatment of cancer, see Yan and Beckman (2005) Biotechniques 39:565-568.

Although considerable research correlating gene expression and/or polymorphisms has been reported, much work remains to be done. This invention supplements the existing body of knowledge and provides related advantages as well.

### SUMMARY OF THE INVENTION

The invention provides compositions and methods for identifying a cancer patient suitable for anti-VEGF therapy. After determining if a patient is likely to be successfully treated, the invention also provides methods for treating the patients.

Thus, in one aspect, this invention provides a method for selecting or identifying a cancer patient suitable for an anti-VEGF therapy comprising, or alternatively consisting essentially of, or yet further consisting of, determining an intratumoral expression level of at least one gene of the group EGFR, VEGFR2 or ERCC1 in a cell or tissue sample of the corresponding cancer isolated from the patient, wherein the presence of:

(a) an EGFR expression level higher than a predetermined first value;

(b) a VEGFR2 expression level higher than a predetermined second value; or

(c) an ERCC1 expression level lower than a predetermined third value,

identifies the patient as suitable for the therapy, or the presence of none of (a) to (c) identifies the patient as not suitable for the therapy. In some embodiments, the presence of:

(d) an EGFR expression level lower than the predetermined first value;

(e) a VEGFR2 expression level lower than the predetermined second value; or

(f) an ERCC1 expression level higher than the predetermined third value,

identifies the patient as not suitable for the therapy.

This invention also provides a method for selecting or identifying a cancer patient suitable for an anti-VEGF therapy comprising, or alternatively consisting essentially of, or yet further consisting of, determining an intratumoral expression level of EGFR in a cell or tissue sample of the corresponding cancer isolated from the patient, wherein a high or overexpression of EGFR or an EGFR expression level higher than a predetermined value identifies the patient as suitable for the therapy, or a low or low expression or an EGFR expression level lower than the predetermined value identifies the patient as not suitable for the therapy.

This invention also provides a method for identifying a cancer patient suitable for or selecting a cancer patient for an anti-VEGF therapy comprising, or alternatively consisting essentially of, or yet further consisting of, determining an intratumoral expression level of VEGFR1 in a cell or tissue sample of the corresponding cancer isolated from the patient, wherein a high or overexpression of VEGFR1 or an VEGFR1 expression level higher than a predetermined value identifies the patient as suitable for the therapy, or a low or underexpression of VEGFR1 or an VEGFR1 expression level lower than the predetermined value identifies the patient as not suitable for the therapy.

Yet further provided is a method for identifying a cancer patient suitable for or selecting a cancer patient for an anti-VEGF therapy comprising, or alternatively consisting essentially of, or yet further consisting of, determining an intratumoral expression level of ERCC1 in a cell or tissue sample of the corresponding cancer isolated from the patient, wherein a low or underexpression of ERCC1 or an ERCC1 expression level lower than a predetermined value identifies the patient as suitable for the therapy, or a high or overexpression of ERCC1 or an ERCC1 expression level higher than the predetermined value identifies the patient as not suitable for the therapy.

This invention also provides a method for treating a cancer patient selected for an anti-VEGF therapy, comprising, or alternatively consisting essentially of, or yet further consisting of, administering to the cancer patient an effective amount of an anti-VEGF therapy, wherein the patient is selected based on one or more of:

(a) an EGFR expression level higher than a predetermined first value;

(b) a VEGFR2 expression level higher than a predetermined second value; or

(c) an ERCC1 expression level lower than a predetermined third value, thereby treating the patient.

The methods are suitable when the cancer patient is suffering from at least one cancer of the type of the group metastatic or non-metastatic rectal cancer, metastatic or non-metastatic colon cancer, metastatic or non-metastatic colorectal cancer, non-small cell lung cancer, metastatic breast cancer, non-metastatic breast cancer, renal cell carcinoma, glioblastoma multiforme, ovarian cancer, hormone-refractory prostate cancer, non-metastatic unresectable liver cancer, head and neck cancer, or metastatic or unresectable locally advanced pancreatic cancer.

Further provided is an anti-VEGF therapy or the use of an anti-VEGF therapy for the therapy of a cancer patient identified for suitable for the therapy using the methods described herein.

Also provided is a kit for use in identifying a cancer patient suitable for a therapy comprising, or alternatively consisting essentially of, or yet further consisting of, suitable primers, probes and/or a microarray for determining a gene expression level for at least one gene of the group EGFR, VEGFR2, or ERCC1, and instructions for use therein.

### DETAILED DESCRIPTION OF THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The practice of the present invention employs, unless otherwise indicated, conventional techniques of molecular biology (including recombinant techniques), microbiology, cell biology, biochemistry and immunology, which are within the skill of the art. Such techniques are explained fully in the literature for example in the following publications. See, e.g., Sambrook and Russell eds. MOLECULAR CLONING: A LABORATORY MANUAL, 3^{rd} edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R. I. Freshney 5^{th} edition (2005)); OLIGONUCLEOTIDE SYNTHESIS (M. J. Gait ed. (1984)); Mullis et al. U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); GENE TRANSFER AND EXPRESSION IN MAMMALIAN CELLS (S. C. Makrides ed. (2003)) IMMUNOCHEMICAL METHODS IN CELL AND MOLECULAR BIOLOGY (Mayer and Walker, eds., Academic Press, London (1987)); WEIR'S HANDBOOK OF EXPERIMENTAL IMMUNOLOGY (L. A. Herzenberg et al. eds (1996)).

### Definitions

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

As used herein, the term “patient” intends an animal, a mammal or yet further a human patient. For the purpose of illustration only, a mammal includes but is not limited to a human, a simian, a murine, a bovine, an equine, a porcine or an ovine.

The term “identify” or “identifying” is to associate or affiliate a patient closely to a group or population of patients who likely experience the same or a similar clinical response to treatment.

As used herein, “anti-VEGF therapy” intends treatment that targets the VEGF receptor family. Without being bound by theory, vascular endothelial growth factor (VEGF) ligands mediate their angiogenic effects by binding to specific VEGF receptors, leading to receptor dimerization and subsequent signal transduction. VEGF ligands bind to 3 primary receptors and 2 co-receptors. Of the primary receptors, VEGFR-1 and VEGFR-2 are mainly associated with angiogenesis. The third primary receptor, VEGFR-3, is associated with lymphangiogenesis.

In one aspect, anti-VEGF therapy comprises, or alternatively consists essentially of, or yet further, consists of an antibody or fragment thereof that binds the VEGF antigen. VEGF (Vascular endothelial growth factor) is a sub-family of growth factors (Entrez Gene: 7422, UniProtKB: P15692 http://www.ncbi.nlm.nih.gov/ last accessed Apr. 17, 2009), more specifically of platelet-derived growth factor family of cystine-knot growth factors. They are important signaling proteins involved in both vasculogenesis (the de novo formation of the embryonic circulatory system) and angiogenesis (the growth of blood vessels from pre-existing vasculature). A non-limiting example of such is the antibody sold under the tradename bevacizumab (abbreviated “BV” herein) or equivalents thereof that bind to the same epitope. It can be polyclonal or monoclonal. The antibody may be of any appropriate species such as for example, murine, ovine or human. It can be humanized, chimeric, recombinant, bispecific, a heteroantibody, a derivative or variant of a polyclonal or monoclonal.

Bevacizumab (BV) is sold under the trade name Avastin by Genentech. It is a humanized monoclonal antibody that binds to and inhibits the biologic activity of human vascular endothelial growth factor (VEGF). Biological equivalent antibodies are identified herein as modified antibodies which bind to the same epitope of the antigen, prevent the interaction of VEGF to its receptors (Flt01, KDR a.k.a. VEGFR2) and produce a substantially equivalent response, e.g., the blocking of endothelial cell proliferation and angiogenesis epitope such as ranibizumab sold under the tradename Lucentis. Bevacizumab is also in the class of cancer drugs that inhibit angiogenesis (angiogenesis inhibitors).

A “native” or “natural” or “wild-type” antigen is a polypeptide, protein or a fragment which contains an epitope and which has been isolated from a natural biological source. It also can specifically bind to an antigen receptor.

As used herein, an “antibody” includes whole antibodies and any antigen binding fragment or a single chain thereof. Thus the term “antibody” includes any protein or peptide containing molecule that comprises at least a portion of an immunoglobulin molecule. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein, any of which can be incorporated into an antibody of the present invention.

If an antibody is used in combination with the above-noted chemotherapy or for diagnosis or as an alternative to the chemotherapy, the antibodies can be polyclonal or monoclonal and can be isolated from any suitable biological source, e.g., murine, rat, sheep and canine. Additional sources are identified infra.

The term “antibody” is further intended to encompass digestion fragments, specified portions, derivatives and variants thereof, including antibody mimetics or comprising portions of antibodies that mimic the structure and/or function of an antibody or specified fragment or portion thereof, including single chain antibodies and fragments thereof. Examples of binding fragments encompassed within the term “antigen binding portion” of an antibody include a Fab fragment, a monovalent fragment consisting of the VL, VH, CL and CH, domains; a F(ab′)₂ fragment, a bivalent fragment comprising two Fab fragments linked by a disulfide bridge at the hinge region; a Fd fragment consisting of the VH and CH, domains; a Fv fragment consisting of the VL and VH domains of a single arm of an antibody, a dAb fragment (Ward et al. (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)). Bird et al. (1988) Science 242:423-426 and Huston et al. (1988) Proc. Natl. Acad. Sci. USA 85:5879-5883. Single chain antibodies are also intended to be encompassed within the term “fragment of an antibody.” Any of the above-noted antibody fragments are obtained using conventional techniques known to those of skill in the art, and the fragments are screened for binding specificity and neutralization activity in the same manner as are intact antibodies.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

In one aspect, the term “equivalent” or “biological equivalent” of an antibody means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody. An example of an equivalent Bevacizumab antibody is one which binds to and inhibits the biologic activity of human vascular endothelial growth factor (VEGF).

The term “antibody variant” is intended to include antibodies produced in a species other than a mouse. It also includes antibodies containing post-translational modifications to the linear polypeptide sequence of the antibody or fragment. It further encompasses fully human antibodies.

The term “antibody derivative” is intended to encompass molecules that bind an epitope as defined above and which are modifications or derivatives of a native monoclonal antibody of this invention. Derivatives include, but are not limited to, for example, bispecific, multispecific, heterospecific, trispecific, tetraspecific, multispecific antibodies, diabodies, chimeric, recombinant and humanized.

The term “bispecific molecule” is intended to include any agent, e.g., a protein, peptide, or protein or peptide complex, which has two different binding specificities. The term “multispecific molecule” or “heterospecific molecule” is intended to include any agent, e.g. a protein, peptide, or protein or peptide complex, which has more than two different binding specificities.

The term “heteroantibodies” refers to two or more antibodies, antibody binding fragments (e.g., Fab), derivatives thereof, or antigen binding regions linked together, at least two of which have different specificities.

The term “human antibody” as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, C_{L}, C_{H} domains (e.g., C_{H1}, C_{H2}, C_{H3}), hinge, (VL, VH)) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

The terms “monoclonal antibody” or “monoclonal antibody composition” as used herein refer to a preparation of antibody molecules of single molecular composition. A monoclonal antibody composition displays a single binding specificity and affinity for a particular epitope.

A “human monoclonal antibody” refers to antibodies displaying a single binding specificity which have variable and constant regions derived from human germline immunoglobulin sequences.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the antibody, e.g., from a transfectoma, antibodies isolated from a recombinant, combinatorial human antibody library, and antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

As used herein, “isotype” refers to the antibody class (e.g., IgM or IgG1) that is encoded by heavy chain constant region genes.

“Platinum drugs” refer to any anticancer compound that includes platinum. In an embodiment, the anticancer drug can be selected from cisplatin (cDDP or cis-iamminedichloroplatinum(II)), carboplatin, oxaliplatin, and combinations thereof.

“Oxaliplatin” (Eloxatin®) is a platinum-based chemotherapy drug in the same family as cisplatin and carboplatin. It is typically administered in combination with fluorouracil and leucovorin in a combination known as FOLFOX for the treatment of colorectal cancer. Compared to cisplatin, the two amine groups are replaced by cyclohexyldiamine for improved antitumour activity. The chlorine ligands are replaced by the oxalato bidentate derived from oxalic acid in order to improve water solubility. Equivalents to Oxaliplatin are known in the art and include, but are not limited to cisplatin, carboplatin, aroplatin, lobaplatin, nedaplatin, and JM-216 (see McKeage et al. (1997) J. Clin. Oncol. 201:1232-1237 and in general, CHEMOTHERAPY FOR GYNECOLOGICAL NEOPLASM, CURRENT THERAPY AND NOVEL APPROACHES, in the Series Basic and Clinical Oncology, Angioli et al. Eds., 2004).

Pyrminidine antimetabolite drug or therapy includes, without limitation fluorouracil (5-FU), which belongs to the family of therapy drugs call pyrimidine based anti-metabolites. 5-FU is a pyrimidine analog, which is transformed into different cytotoxic metabolites that are then incorporated into DNA and RNA thereby inducing cell cycle arrest and apoptosis. Chemical equivalents are pyrimidine analogs which result in disruption of DNA replication. Chemical equivalents inhibit cell cycle progression at S phase resulting in the disruption of cell cycle and consequently apoptosis. Equivalents to 5-FU include prodrugs, analogs and derivative thereof such as 5′-deoxy-5-fluorouridine (doxifluoroidine), 1-tetrahydrofuranyl-5-fluorouracil (ftorafur), Capecitabine (Xeloda), S-1 (MBMS-247616, consisting of tegafur and two modulators, a 5-chloro-2,4-dihydroxypyridine and potassium oxonate), ralititrexed (tomudex), nolatrexed (Thymitaq, AG337), LY231514 and ZD9331, as described for example in Papamicheal (1999) The Oncologist 4:478-487. For the purpose of this invention, pyrmidine antimetabolite drugs includes 5-FU based adjuvant therapy.

Fluorouracil (5-FU) belongs to the family of therapy drugs call pyrimidine based anti-metabolites. It is a pyrimidine analog, which is transformed into different cytotoxic metabolites that are then incorporated into DNA and RNA thereby inducing cell cycle arrest and apoptosis. Chemical equivalents are pyrimidine analogs which result in disruption of DNA replication. Chemical equivalents inhibit cell cycle progression at S phase resulting in the disruption of cell cycle and consequently apoptosis. Equivalents to 5-FU include prodrugs, analogs and derivative thereof such as 5′-deoxy-5-fluorouridine (doxifluoroidine), 1-tetrahydrofuranyl-5-fluorouracil (ftorafur), Capecitabine (Xeloda), S-1 (MBMS-247616, consisting of tegafur and two modulators, a 5-chloro-2,4-dihydroxypyridine and potassium oxonate), ralititrexed (tomudex), nolatrexed (Thymitaq, AG337), LY231514 and ZD9331, as described for example in Papamicheal (1999) The Oncologist 4:478-487.

Capecitabine is a prodrug of (5-FU) that is converted to its active form by the tumor-specific enzyme PynPase following a pathway of three enzymatic steps and two intermediary metabolites, 5′-deoxy-5-fluorocytidine (5′-DFCR) and 5′-deoxy-5-fluorouridine (5′-DFUR). Capecitabine is marketed by Roche under the trade name Xeloda®.

Leucovorin (Folinic acid) is an adjuvant used in cancer therapy. It is used in synergistic combination with 5-FU to improve efficacy of the chemotherapeutic agent. Without being bound by theory, addition of Leucovorin is believed to enhance efficacy of 5-FU by inhibiting thymidylate synthase. It has been used as an antidote to protect normal cells from high doses of the anticancer drug methotrexate and to increase the antitumor effects of fluorouracil (5-FU) and tegafur-uracil. It is also known as citrovorum factor and Wellcovorin. This compound has the chemical designation of L-Glutamic acid N[4[[(2-amino-5-formyl-1,4,5,6,7,8hexahydro-4-oxo6-pteridinyl)methyl]amino]benzoyl], calcium salt (1:1).

“FOLFOX” is an abbreviation for a type of combination therapy that is used to treat cancer. This therapy includes 5-FU, oxaliplatin and leucovorin. “FOLFIRI” is an abbreviation for a type of combination therapy that is used treat cancer and comprises, or alternatively consists essentially of, or yet further consists of 5-FU, leucovorin, and irinotecan. Information regarding these treatments is available on the National Cancer Institute's web site, cancer.gov, last accessed on Jan. 16, 2008. Equivalents of FOLFOX/BV intend where one or more of the components of the composition are substituted with an equivalent, e.g., an equivalent to 5-FU and/or oxaliplatin.

“XELOX/BV” is another combination therapy used to treat colorectal cancer, which includes the prodrug to 5-FU, known as Capecitabine (Xeloda) in combination with oxaliplatin and bevacizumab. Equivalents of XELOX/BV intend where one or more of the components of the composition are substituted with an equivalent, e.g., an equivalent to bevacizumab and/or oxaliplatin. Information regarding these treatments is available on the National Cancer Institute's web site, cancer.gov or from the National Comprehensive Cancer Network's web site, nccn.org, last accessed on May 27, 2008.

The term “adjuvant” cancer patient refers to a patient to which administration of a therapy or chemotherapeutic regimen has been given after removal of a tumor by surgery, usually termed adjuvant chemotherapy. Adjuvant therapy is typically given to minimize or prevent a possible cancer reoccurrence. Alternatively, “neoadjuvant” therapy refers to administration of therapy or chemotherapeutic regimen before surgery, typically in an attempt to shrink the tumor prior to a surgical procedure to minimize the extent of tissue removed during the procedure.

The phrase “first line” or “second line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as primary therapy and primary treatment.” See National Cancer Institute website as www.cancer.gov, last visited on May 1, 2008. Typically, a patient is given a subsequent chemotherapy regimen because the patient did not shown a positive clinical or sub-clinical response to the first line therapy or the first line therapy has stopped.

In one aspect, the term “equivalent” of “chemical equivalent” of a chemical means the ability of the chemical to selectively interact with its target protein, DNA, RNA or fragment thereof as measured by the inactivation of the target protein, incorporation of the chemical into the DNA or RNA or other suitable methods. Chemical equivalents include, but are not limited to, those agents with the same or similar biological activity and include, without limitation a pharmaceutically acceptable salt or mixtures thereof that interact with and/or inactivate the same target protein, DNA, or RNA as the reference chemical.

The term “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions and insertions of nucleotides. An allele of a gene can also be a form of a gene containing a mutation.

The term “genetic marker” refers to an allelic variant of a polymorphic region of a gene of interest and/or the expression level of a gene of interest.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic region can be a single nucleotide, the identity of which differs in different alleles.

A “polymorphic gene” refers to a gene having at least one polymorphic region.

A “haplotype” is a set of alleles of a group of closely linked genes which are usually inherited as a unit. The term “allelic variant of a polymorphic region of the gene of interest” refers to a region of the gene of interest having one of a plurality of nucleotide sequences found in that region of the gene in other individuals.

The term “genotype” refers to the specific allelic composition of an entire cell or a certain gene and in some aspects a specific polymorphism associated with that gene, whereas the term “phenotype’ refers to the detectable outward manifestations of a specific genotype.

An “internal control” or “house keeping” gene refers to any constitutively or globally expressed gene. Examples of such genes include, but are not limited to, β-actin, the transferring receptor gene, GAPDH gene or equivalents thereof. In one aspect of the invention, the internal control gene is β-actin.

“Overexpression” or “underexpression” refers to increased or decreased expression, or alternatively a differential expression, of a gene in a test sample as compared to the expression level of that gene in the control sample. In one aspect, the test sample is a diseased cell, and the control sample is a normal cell. In another aspect, the test sample is an experimentally manipulated or biologically altered cell, and the control sample is the cell prior to the experimental manipulation or biological alteration. In yet another aspect, the test sample is a sample from a patient, and the control sample is a similar sample from a healthy individual. In a yet further aspect, the test sample is a sample from a patient and the control sample is a similar sample from patient not having the desired clinical outcome. In one aspect, the differential expression is about 1.5 times, or alternatively, about 2.0 times, or alternatively, about 2.0 times, or alternatively, about 3.0 times, or alternatively, about 5 times, or alternatively, about 10 times, or alternatively about 50 times, or yet further alternatively more than about 100 times higher or lower than the expression level detected in the control sample. Alternatively, the gene is referred to as “over expressed” or “under expressed”. Alternatively, the gene may also be referred to as “up regulated” or “down regulated”.

A “predetermined value” for a gene as used herein, is so chosen that a patient with an expression level of that gene higher than the predetermined value is likely to experience a more or less desirable clinical outcome than patients with expression levels of the same gene lower than the predetermined value, or vice-versa. Expression levels of genes, such as those disclosed in the present invention, are associated with clinical outcomes. One of skill in the art can determine a predetermined value for a gene by comparing expression levels of a gene in patients with more desirable clinical outcomes to those with less desirable clinical outcomes. In one aspect, a predetermined value is a gene expression value that best separates patients into a group with more desirable clinical outcomes and a group with less desirable clinical outcomes. Such a gene expression value can be mathematically or statistically determined with methods well known in the art.

Alternatively, a gene expression that is higher than the predetermined value is simply referred to as a “high expression”, or a gene expression that is lower than the predetermined value is simply referred to as a “low expression”.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The phrase “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu, D. Y. et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “mismatches” refers to hybridized nucleic acid duplexes which are not 100% homologous. The lack of total homology may be due to deletions, insertions, inversions, substitutions or frameshift mutations.

The term “isolated” as used herein refers to molecules or biological or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

When the expression level of a gene or a genetic marker or polymorphism is used as a basis for selecting a patient for a treatment described herein, the expression level or genetic marker or polymorphism is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of the genetic marker or polymorphism in a clinical setting is a clear indication that this parameter was used as a basis for initiating, continuing, adjusting and/or ceasing administration of the treatments described herein.

The term “treating” as used herein is intended to encompass curing as well as ameliorating at least one symptom of the condition or disease. For example, in the case of cancer, a response to treatment includes a reduction in cachexia, increase in survival time, elongation in time to tumor progression, reduction in tumor mass, reduction in tumor burden and/or a prolongation in time to tumor metastasis, time to tumor recurrence, tumor response, complete response, partial response, stable disease, progressive disease, progression free survival, overall survival, each as measured by standards set by the National Cancer Institute and the U.S. Food and Drug Administration for the approval of new drugs. See Johnson et al. (2003) J. Clin. Oncol. 21(7):1404-1411.

“An effective amount” intends to indicate the amount of a compound or agent administered or delivered to the patient which is most likely to result in the desired response to treatment. The amount is empirically determined by the patient's clinical parameters including, but not limited to the Stage of disease, age, gender, histology, and likelihood for tumor recurrence.

The term “clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), relative risk (RR), toxicity or side effect.

The term “likely to respond” intends to mean that the patient of a genotype is relatively more likely to experience a complete response or partial response than patients similarly situated without the genotype. Alternatively, the term “not likely to respond” intends to mean that the patient of a genotype is relatively less likely to experience a complete response or partial response than patients similarly situated without the genotype.

The term “suitable for a therapy” or “suitably treated with a therapy” shall mean that the patient is likely to exhibit one or more more desirable clinical outcome as compared to patients having the same disease and receiving the same therapy but possessing a different characteristic that is under consideration for the purpose of the comparison. In one aspect, the characteristic under consideration is a genetic polymorphism or a somatic mutation. In another aspect, the characteristic under consideration is expression level of a gene or a polypeptide. In one aspect, a more desirable clinical outcome is relatively higher likelihood of or relatively better tumor response such as tumor load reduction. In another aspect, a more desirable clinical outcome is relatively longer overall survival. In yet another aspect, a more desirable clinical outcome is relatively longer progression free survival or time to tumor progression. In yet another aspect, a more desirable clinical outcome is relatively longer disease free survival. In further another aspect, a more desirable clinical outcome is relative reduction or delay in tumor recurrence. In another aspect, a more desirable clinical outcome is relatively decreased metastasis. In another aspect, a more desirable clinical outcome is relatively lower relative risk. In yet another aspect, a more desirable clinical outcome is relatively reduced toxicity or side effects. In some embodiments, more than one clinical outcomes are considered simultaneously. In one such aspect, a patient possessing a characteristic, such as a genotype of a genetic polymorphism, may exhibit more than one more desirable clinical outcomes as compared to patients having the same disease and receiving the same therapy but not possessing the characteristic. As defined herein, the patients is considered suitable for the therapy. In another such aspect, a patient possessing a characteristic may exhibit one or more more desirable clinical outcome but simultaneously exhibit one or more less desirable clinical outcome. The clinical outcomes will then be considered collectively, and a decision as to whether the patient is suitable for the therapy will be made accordingly, taking into account the patient's specific situation and the relevance of the clinical outcomes. In some embodiments, progression free survival or overall survival is weighted more heavily than tumor response in a collective decision making

A “complete response” (CR) to a therapy defines patients with evaluable but non-measurable disease, whose tumor and all evidence of disease had disappeared.

A “partial response” (PR) to a therapy defines patients with anything less than complete response that were simply categorized as demonstrating partial response.

“Stable disease” (SD) indicates that the patient is stable.

“Progressive disease” (PD) indicates that the tumor has grown (i.e. become larger), spread (i.e. metastasized to another tissue or organ) or the overall cancer has gotten worse following treatment. For example, tumor growth of more than 20 percent since the start of treatment typically indicates progressive disease. “Disease free survival” indicates the length of time after treatment of a cancer or tumor during which a patient survives with no signs of the cancer or tumor.

“Non-response” (NR) to a therapy defines patients whose tumor or evidence of disease has remained constant or has progressed.

“Overall Survival” (OS) intends a prolongation in life expectancy as compared to naïve or untreated individuals or patients.

“Progression free survival” (PFS) or “Time to Tumor Progression” (TTP) indicates the length of time during and after treatment that the cancer does not grow. Progression-free survival includes the amount of time patients have experienced a complete response or a partial response, as well as the amount of time patients have experienced stable disease.

“No Correlation” refers to a statistical analysis showing no relationship between the allelic variant of a polymorphic region or gene expression levels and clinical parameters.

“Tumor Recurrence” as used herein and as defined by the National Cancer Institute is cancer that has recurred (come back), usually after a period of time during which the cancer could not be detected. The cancer may come back to the same place as the original (primary) tumor or to another place in the body. It is also called recurrent cancer.

“Time to Tumor Recurrence” (TTR) is defined as the time from the date of diagnosis of the cancer to the date of first recurrence, death, or until last contact if the patient was free of any tumor recurrence at the time of last contact. If a patient had not recurred, then TTR was censored at the time of death or at the last follow-up.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

As used herein, the terms “Stage I cancer,” “Stage II cancer,” “Stage III cancer,” and “Stage IV” refer to the TNM staging classification for cancer. Stage I cancer typically identifies that the primary tumor is limited to the organ of origin. Stage II intends that the primary tumor has spread into surrounding tissue and lymph nodes immediately draining the area of the tumor. Stage III intends that the primary tumor is large, with fixation to deeper structures. Stage IV intends that the primary tumor is large, with fixation to deeper structures. See pages 20 and 21, CANCER BIOLOGY, 2^{nd} Ed., Oxford University Press (1987).

“Having the same cancer” is used when comparing one patient to another or alternatively, one patient population to another patient population. For example, the two patients or patient populations will each have or be suffering from colon cancer.

A “tumor” is an abnormal growth of tissue resulting from uncontrolled, progressive multiplication of cells and serving no physiological function. A “tumor” is also known as a neoplasm.

The term “blood” refers to blood which includes all components of blood circulating in a subject including, but not limited to, red blood cells, white blood cells, plasma, clotting factors, small proteins, platelets and/or cryoprecipitate. This is typically the type of blood which is donated when a human patent gives blood.

### Descriptive Embodiments

The invention further provides diagnostic, prognostic and therapeutic methods, which are based, at least in part, on determination of the expression level of a gene of interest identified herein.

For example, information obtained using the diagnostic assays described herein is useful for determining if a subject is suitable for cancer treatment of a given type. Based on the prognostic information, a doctor can recommend a therapeutic protocol, useful for reducing the malignant mass or tumor in the patient or treat cancer in the individual.

Determining whether a subject is suitable or not suitable for cancer treatment of a given type, alternatively, can be expressed as identifying a subject suitable for the cancer treatment or identifying a subject not suitable for the cancer treatment of the given type.

It is to be understood that information obtained using the diagnostic assays described herein may be used alone or in combination with other information, such as, but not limited to, genotypes or expression levels of other genes, clinical chemical parameters, histopathological parameters, or age, gender and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient and etc. In a particular aspect, the genotypes or expression levels of one or more genes as disclosed herein are used in a panel of genes, each of which contributes to the final diagnosis, prognosis or treatment.

The methods of this invention are useful for the diagnosis, prognosis and treatment of patients suffering from at least one or more cancer of the group: metastatic or non-metastatic rectal cancer, metastatic or non-metastatic colon cancer, metastatic or non-metastatic colorectal cancer, lung cancer, head and neck cancer, non-small cell lung cancer, metastatic breast cancer, non-metastatic breast cancer, renal cell carcinoma, glioblastoma multiforme, ovarian cancer, hormone-refractory prostate cancer, non-metastatic unresectable liver cancer, or metastatic or unresectable locally advanced pancreatic cancer.

The methods are useful in the assistance of an animal, a mammal or yet further a human patient. For the purpose of illustration only, a mammal includes but is not limited to a simian, a murine, a bovine, an equine, a porcine or an ovine.

### Diagnostic Methods

The invention further provides diagnostic methods, which are based, at least in part, on determination of the expression level of a gene of interest identified herein. Thus, in one aspect, this invention provides a method for identifying a cancer patient suitable or not suitable for an anti-VEGF therapy comprising, or alternatively consisting essentially of, or yet further consisting of, determining an intratumoral expression level of at least one gene of the group EGFR, VEGFR2 or ERCC1 in a cell or tissue sample of the corresponding cancer isolated from the patient, wherein the presence of:

(a) an EGFR expression level higher than a predetermined first value;

(b) a VEGFR2 expression level higher than a predetermined second value; or

(c) an ERCC1 expression level lower than a predetermined third value,

identifies the patient as suitable for the therapy, or the presence of none of (a) to (c) identifies the patient as not suitable for the therapy. In some embodiments, the presence of:

(d) an EGFR expression level lower than the predetermined first value;

(e) a VEGFR2 expression level lower than the predetermined second value; or

(f) an ERCC1 expression level higher than the predetermined third value,

identifies the patient as not suitable for the therapy.

In another aspect, this invention provides a method for identifying a cancer patient suitable or not suitable for an anti-VEGF therapy comprising, or alternatively consisting essentially of, or yet further consisting of, determining an intratumoral expression level of at least one gene of the group EGFR, VEGFR2 or ERCC1 in a cell or tissue sample of the corresponding cancer isolated from the patient, wherein the presence of:

(a) an EGFR expression level higher than a predetermined first value;

(b) a VEGFR2 expression level higher than a predetermined second value; or

(c) an ERCC1 expression level lower than a predetermined third value,

identifies the patient as suitable for the therapy, or the presence of none of (a) to (c) identifies the patient as not suitable for the therapy. In some embodiments, the presence of:

(d) an EGFR expression level lower than the predetermined first value;

(e) a VEGFR2 expression level lower than the predetermined second value; or

(f) an ERCC1 expression level higher than the predetermined third value, identifies the patient as not suitable for the therapy.

Thus in one aspect, the patient identified as suitable for the therapy has an EGFR expression level higher than the predetermined first value, a VEGFR2 expression level higher than the predetermined second value, or an ERCC1 expression level lower than the predetermined third value identifies the patient as suitable for the therapy. Alternatively, a high or overexpression of EGFR or VEGFR2, or a low or underexpression of ERCC1, identifies the patient as suitable for the therapy.

The patient is suitable for the therapy because they are more likely to experience a longer progress free survival than patients identified as not having the genotype and having the same cancer and receiving the same anti-VEGF therapy.

In another aspect, the patient is identified as not suitable for the therapy when an EGFR expression level lower than the predetermined first value, a VEGFR2 expression level lower than the predetermined second value, or an ERCC1 expression level higher than the predetermined third value identifies the patient as not suitable for the therapy. Alternatively, a low or underexpression of EGFR or VEGFR2, or a high or overexpression of ERCC1, identifies the patient as suitable for the therapy. The patient is not suitable for the therapy because they are less likely to experience a longer progress free survival than patients identified as not having the expression level and having the same cancer and receiving the same anti-VEGF therapy.

Also provided is a method for identifying a cancer patient suitable or not suitable for an anti-VEGF therapy comprising, or alternatively consisting essentially of, or yet further consisting of, determining an intratumoral expression level of ERCC1 in a cell or tissue sample of the corresponding cancer isolated from the patient, wherein a low or underexpression of ERCC1 or an ERCC1 expression level lower than a predetermined value identifies the patient as suitable for the therapy, or a high or overexpression of ERCC1 or an ERCC1 expression level higher than the predetermined value identifies the patient as not suitable for the therapy.

In one aspect, a method is provided for determining if a cancer patient is suitable or is not suitable for an anti-VEGF therapy comprising, or alternatively consisting essentially of, or consisting of, determining an intratumoral expression level of EGFR in a cell or tissue sample of the corresponding cancer isolated from the patient, wherein a high or overexpression of EGFR or an EGFR expression level higher than a predetermined value identifies the patient as suitable for the therapy, or a low or an underexpression of EGFR or an EGFR expression level lower than the predetermined value identifies the patient as not suitable for the therapy. Patients suitable for the therapy are more likely to experience a longer progress free survival than patients having a low our underexpression of EGFR or an EGFR expression level lower than the predetermined value and having the cancer and receiving the anti-VEGF therapy. Patients not suitable for the therapy are less likely to experience a longer progress free survival than patients not having the expression level and having the cancer and receiving the anti-VEGF therapy.

This invention also provides a method for identifying a cancer patient suitable or not suitable for an anti-VEGF therapy comprising, or alternatively consisting essentially of, or yet further consisting of, determining an intratumoral expression level of VEGFR1 in a cell or tissue sample of the corresponding cancer isolated from the patient, wherein a high or overexpression of VEGFR1 or an VEGFR1 expression level higher than a predetermined value identifies the patient as suitable for the therapy, or a low or an underexpression of VEGFR1 or an VEGFR1 expression level lower than the predetermined value identifies the patient as not suitable for the therapy. Patients suitable for the therapy are more likely to experience a longer progress free survival than patients having a low or underexpression of EGFR or an EGFR expression level lower than the predetermined value and having the cancer and receiving the anti-VEGF therapy. Patients not suitable for the therapy are less likely to experience a longer progress free survival than patients not having the expression level and having the cancer and receiving the anti-VEGF therapy.

The anti-VEGF therapy comprises, or alternatively consists essentially of, or yet further consists of administration of an anti-VEGF therapy, which in one aspect comprises, or alternatively consists essentially of, or yet further consists of administration of an anti-VEGF antibody or an equivalent thereof. Bevacizumab or an equivalent thereof are examples of anti-VEGF antibody therapy. In another aspect, the therapy comprises, or alternatively consists essentially of, or yet further consists of, administration of a platinum drug as defined herein, which includes for example, oxaliplatin or an equivalent thereof. In a yet further aspect, the therapy further comprises, or alternatively consists essentially of, or yet further consists of, administration of a pyrimidine antimetabolite such as 5-FU or capecitabine or equivalents thereof. Examples of such therapies include but are not limited to administration FOLFOX/BV (5-FU, leucovorin, oxaliplatin, and bevacizumab) or an equivalent thereof; XELOX/BV (capecitabine, leucovorin, oxaliplatin, and bevacizumab) or an equivalent thereof; the administration of bevacizumab or an equivalent thereof, and oxaliplatin or an equivalent thereof, and/or 5-FU or capecitabine or equivalents thereof. The administrations can be concurrent or sequential. The therapies can be first line, second line or third line therapies. In one particular aspect, the anti-VEGF therapy is a first line therapy.

In one aspect, the cancer patient is suffering from at least one cancer of the type of the group metastatic or non-metastatic rectal cancer, metastatic or non-metastatic colon cancer, metastatic or non-metastatic colorectal cancer, non-small cell lung cancer, metastatic breast cancer, non-metastatic breast cancer, renal cell carcinoma, glioblastoma multiforme, ovarian cancer, hormone-refractory prostate cancer, non-metastatic unresectable liver cancer, or metastatic or unresectable locally advanced pancreatic cancer. In another aspect, the cancer patient is suffering from colorectal cancer. In yet a further aspect, the cancer patient is suffering from metastatic colorectal cancer.

The patient is selected for the therapy by determining from a suitable patient sample at least one or more of:

(a) an EGFR expression level higher than a predetermined first value;

(b) a VEGFR2 expression level higher than a predetermined second value; or

(c) an ERCC1 expression level lower than a predetermined third value,

in a sample isolated from the patient. The sample is at least one of a tumor or cancer cell sample which can be a fixed tissue, a frozen tissue, a biopsy tissue, a resection tissue, a microdissected tissue, or combinations thereof. For the purpose of this method, the patient is an animal patient, e.g., a mammalian, simian, bovine, murine, equine, porcine or ovine patient. In another aspect, the patient is a human patient.

Methods of determining gene expression levels are known in the art. For the purpose of illustration only, such methods can include determining the amount of a mRNA transcribed from the gene using, for example, a method comprising, or alternatively consisting essentially of, or yet further consisting of, one or more of in situ hybridization, PCR, real-time PCR, or microarray. The methods can be performed on at least one of a fixed tissue, a frozen tissue, a biopsy tissue, a resection tissue, a microdissected tissue, or combinations thereof.

In addition, knowledge of the identity of the expression level of a gene in an individual (the gene profile) allows customization of therapy for a particular disease to the individual's genetic profile, the goal of “pharmacogenomics”. For example, an individual's genetic profile can enable a doctor: 1) to more effectively prescribe a drug that will address the molecular basis of the disease or condition; 2) to better determine the appropriate dosage of a particular drug and 3) to identify novel targets for drug development. The identity of the genotype or expression patterns of individual patients can then be compared to the genotype or expression profile of the disease to determine the appropriate drug and dose to administer to the patient.

The ability to target populations expected to show the highest clinical benefit, based on the normal or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling.

The methods described herein may be performed, for example, by utilizing pre-packaged diagnostic kits, such as those described below, comprising at least one probe or primer nucleic acid described herein, which may be conveniently used, e.g., to determine whether a subject is likely to experience tumor recurrence following therapy as described herein or has or is at risk of developing disease such as colon cancer.

Sample nucleic acid for use in the above-described diagnostic and prognostic methods can be obtained from any suitable cell type or tissue of a subject. For example, a subject's bodily fluid (e.g. blood) can be obtained by known techniques (e.g., venipuncture). Alternatively, nucleic acid tests can be performed on dry samples (e.g., hair or skin). Diagnostic procedures can also be performed in situ directly upon tissue sections (fixed and/or frozen) of patient tissue obtained from biopsies or resections, such that no nucleic acid purification is necessary. Nucleic acid reagents can be used as probes and/or primers for such in situ procedures (see, for example, Nuovo, G. J. (1992) PCR IN SITU HYBRIDIZATION: PROTOCOLS AND APPLICATIONS, RAVEN PRESS, NY).

In addition to methods which focus primarily on the detection of one nucleic acid sequence, profiles can also be assessed in such detection schemes. Fingerprint profiles can be generated, for example, by utilizing a differential display procedure, Northern analysis and/or RT-PCR.

Antibodies directed against wild type or mutant peptides encoded by the allelic variants of the gene of interest may also be used in disease diagnostics and prognostics. Such diagnostic methods, may be used to detect abnormalities in the level of expression of the peptide, or abnormalities in the structure and/or tissue, cellular, or subcellular location of the peptide. Protein from the tissue or cell type to be analyzed may easily be detected or isolated using techniques which are well known to one of skill in the art, including but not limited to Western blot analysis. For a detailed explanation of methods for carrying out Western blot analysis, see Sambrook and Russell (2001) supra. The protein detection and isolation methods employed herein can also be such as those described in Harlow and Lane, (1999) supra. This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of the peptides or their allelic variants. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the subject polypeptide, but also its distribution in the examined tissue. Using the present invention, one of ordinary skill will readily perceive that any of a wide variety of histological methods (such as staining procedures) can be modified in order to achieve such in situ detection.

Probes can be affixed to surfaces for use as “gene chips.” Such gene chips can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley, S. O. et al. (1999) Nucleic Acids Res. 27:4830-4837.

This invention also provides for a prognostic panel of genetic markers selected from, but not limited to the probes and/or primers to determine gene expression as identified herein. The probes or primers can be attached or supported by a solid phase support such as, but not limited to a gene chip or microarray. The probes or primers can be detectably labeled. In one aspect, provided is a panel of probes and/or primers to determine an intratumoral expression level of at least two genes of the group EGFR, VEGFR2 or ERCC1 in a cell or tissue sample.

In one aspect, the panel contains the herein identified probes or primers as wells as other probes or primers. In a alternative aspect, the panel includes one or more of the above noted probes or primers and others. In a further aspect, the panel consist only of the above-noted probes or primers.

Primers or probes can be affixed to surfaces for use as “gene chips” or “microarray.” Such gene chips or microarrays can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

Various “gene chips” or “microarray” and similar technologies are know in the art.

Examples of such include, but are not limited to LabCard (ACLARA Bio Sciences Inc.); GeneChip (Affymetric, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc.); GEM (Incyte Microarray Systems); a high-throughput microarraying system that can dispense from 12 to 64 spots onto multiple glass slides (Intelligent Bio-Instruments); Molecular Biology Workstation and NanoChip (Nanogen, Inc.); a microfluidic glass chip (Orchid biosciences, Inc.); BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu Rev. Biomed. Eng. 4:129-153. Examples of “Gene chips” or a “microarray” are also described in U.S. Patent Publ. Nos.: 2007/0111322, 2007/0099198, 2007/0084997, 2007/0059769 and 2007/0059765 and U.S. Pat. Nos. 7,138,506, 7,070,740, and 6,989,267.

In one aspect, “gene chips” or “microarrays” containing probes or primers for the gene of interest are provided alone or in combination with other probes and/or primers. A suitable sample is obtained from the patient extraction of genomic DNA, RNA, or any combination thereof and amplified if necessary. The DNA or RNA sample is contacted to the gene chip or microarray panel under conditions suitable for hybridization of the gene(s) of interest to the probe(s) or primer(s) contained on the gene chip or microarray. The probes or primers may be detectably labeled thereby identifying the polymorphism in the gene(s) of interest. Alternatively, a chemical or biological reaction may be used to identify the probes or primers which hybridized with the DNA or RNA of the gene(s) of interest. The genetic profile of the patient is then determined with the aid of the aforementioned apparatus and methods.

### Nucleic Acids

In one aspect, the nucleic acid sequences of the gene of interest, or portions thereof, can be the basis for probes or primers, e.g., in methods for determining expression level of the gene of interest or the allelic variant of a polymorphic region of a gene of interest identified in the experimental section below. Thus, they can be used in the methods of the invention to determine which therapy is most likely to treat an individual's cancer.

The methods of the invention can use nucleic acids isolated from vertebrates. In one aspect, the vertebrate nucleic acids are mammalian nucleic acids. In a further aspect, the nucleic acids used in the methods of the invention are human nucleic acids.

Primers for use in the methods of the invention are nucleic acids which hybridize to a nucleic acid sequence which is adjacent to the region of interest or which covers the region of interest and is extended. A primer can be used alone in a detection method, or a primer can be used together with at least one other primer or probe in a detection method. Primers can also be used to amplify at least a portion of a nucleic acid. Probes for use in the methods of the invention are nucleic acids which hybridize to the gene of interest and which are not further extended. For example, a probe is a nucleic acid which hybridizes to the gene of interest, and which by hybridization or absence of hybridization to the DNA of a subject will be indicative of the identity of the allelic variant of the expression levels of the gene of interest. Primers and/or probes for use in the methods can be provided as isolated single stranded oligonucleotides or alternatively, as isolated double stranded oligonucleotides.

In one embodiment, primers comprise a nucleotide sequence which comprises a region having a nucleotide sequence which hybridizes under stringent conditions to about: 6, or alternatively 8, or alternatively 10, or alternatively 12, or alternatively 25, or alternatively 30, or alternatively 40, or alternatively 50, or alternatively 75 consecutive nucleotides of the gene of interest.

Primers can be complementary to nucleotide sequences located close to each other or further apart, depending on the use of the amplified DNA. For example, primers can be chosen such that they amplify DNA fragments of at least about 10 nucleotides or as much as several kilobases. Preferably, the primers of the invention will hybridize selectively to nucleotide sequences located about 100 to about 1000 nucleotides apart.

For amplifying at least a portion of a nucleic acid, a forward primer (i.e., 5′ primer) and a reverse primer (i.e., 3′ primer) will preferably be used. Forward and reverse primers hybridize to complementary strands of a double stranded nucleic acid, such that upon extension from each primer, a double stranded nucleic acid is amplified.

Yet other preferred primers of the invention are nucleic acids which are capable of selectively hybridizing to the TS gene. Thus, such primers can be specific for the gene of interest sequence, so long as they have a nucleotide sequence which is capable of hybridizing to the gene of interest.

The probe or primer may further comprises a label attached thereto, which, e.g., is capable of being detected, e.g. the label group is selected from amongst radioisotopes, fluorescent compounds, enzymes, and enzyme co-factors.

Additionally, the isolated nucleic acids used as probes or primers may be modified to become more stable. Exemplary nucleic acid molecules which are modified include phosphoramidate, phosphothioate and methylphosphonate analogs of DNA (see also U.S. Pat. Nos. 5,176,996; 5,264,564 and 5,256,775).

The nucleic acids used in the methods of the invention can also be modified at the base moiety, sugar moiety, or phosphate backbone, for example, to improve stability of the molecule. The nucleic acids, e.g., probes or primers, may include other appended groups such as peptides (e.g., for targeting host cell receptors in vivo), or agents facilitating transport across the cell membrane. See, e.g., Letsinger et al. (1989) Proc. Natl. Acad. Sci. U.S.A. 86:6553-6556; Lemaitre et al. (1987) Proc. Natl. Acad. Sci. 84:648-652; and PCT Publ. No. WO 88/09810, published Dec. 15, 1988), hybridization-triggered cleavage agents, (see, e.g., Krol et al. (1988) BioTechniques 6:958-976) or intercalating agents (see, e.g., Zon (1988) Pharm. Res. 5:539-549. To this end, the nucleic acid used in the methods of the invention may be conjugated to another molecule, e.g., a peptide, hybridization triggered cross-linking agent, transport agent, hybridization-triggered cleavage agent, etc.

The isolated nucleic acids used in the methods of the invention can also comprise at least one modified sugar moiety selected from the group including but not limited to arabinose, 2-fluoroarabinose, xylulose, and hexose or, alternatively, comprise at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

The nucleic acids, or fragments thereof, to be used in the methods of the invention can be prepared according to methods known in the art and described, e.g., in Sambrook et al. (2001) supra. For example, discrete fragments of the DNA can be prepared and cloned using restriction enzymes. Alternatively, discrete fragments can be prepared using the Polymerase Chain Reaction (PCR) using primers having an appropriate sequence under the manufacturer's conditions, (described above).

Oligonucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al. (1988) Nucl. Acids Res. 16:3209, methylphosphonate oligonucleotides can be prepared by use of controlled pore glass polymer supports. Sarin et al. (1988) Proc. Natl. Acad. Sci. U.S.A. 85:7448-7451.

### Methods of Treatment

This invention also provides a method for treating a cancer patient selected for therapy based on the presence of a genotype as described above, comprising, or alternatively consisting essentially of, or yet further consisting of, administering an effective amount of an anti-VEGF therapy to the patient, wherein the patient was identified by a method described above, thereby treating the patient.

Also provided is a method for treating a cancer patient, comprising administering an anti-VEGF therapy to a cancer patient selected for the therapy based on one or more of:

(a) an EGFR expression level higher than a predetermined first value,

(b) a VEGFR2 expression level higher than a predetermined second value, or

(c) an ERCC1 expression level lower than a predetermined third value,

in a sample isolated from the patient, thereby treating the patient.

In one aspect, the patient is selected by a method comprising determining an intratumoral expression level of at least one gene of the group EGFR, VEGFR2 or ERCC1 in a cell or tissue sample of the corresponding cancer isolated from the patient.

The invention further provides methods for treating patients having solid malignant tissue mass or tumor selected for or identified as being suitable for the treatment. In one aspect, a patient is selected or suitable if he or she is more likely to respond to the anti-VEGF therapy than another patient receiving the same therapy and having the same cancer but not identified or determined to be suitable for the therapy. In one aspect, a patient is selected or suitable for the therapy if he experiences a relatively longer progression free survival or overall survival than a patient having the same cancer and receiving the same therapy but not identified or determined to be suitable for the anti-VEGF therapy.

For the purpose of these methods, the anti-VEGF therapy comprises, or alternatively consists essentially of, or yet further consisting of administration of one or more of an anti-VEGF antibody or an equivalent thereof. In another aspect, the anti-VEGF therapy comprises, or alternatively consists essentially of, or yet further consists of administration of bevacizumab or an equivalent thereof. In a further aspect, the anti-VEGF therapy further comprises, or alternatively consists essentially of, or consists of administration of a platinum drug. In a yet further aspect, the platinum drug is oxaliplatin or an equivalent thereof. In an alternative aspect, the anti-VEGF therapy further comprises, or alternatively consists essentially of, or alternatively consists of administration of a pyrimidine antimetabolite drug. In a yet further aspect, the pyrimidine antimetabolite drug is 5-FU, capecitabine, or equivalents thereof. In another aspect, the anti-VEGF therapy comprises, or alternatively consists essentially of, or alternatively consists of administration of an anti-VEGF antibody in combination with a platinum drug and a pyrimidine antimetabolite drug. In another aspect, the anti-VEGF therapy comprises, or alternatively consists essentially of, or yet further consists of, administration of one or more of bevacizumab or an equivalent thereof in combination with oxaliplatin or an equivalent thereof, and 5-FU, capecitabine, or equivalents thereof. In another aspect, the anti-VEGF therapy comprises, or alternatively consists essentially of, or alternatively consists of, administration of FOLFOX/BV (5-FU, leucovorin, oxaliplatin, and bevacizumab) or an equivalent thereof, or XELOX/BV (capecitabine, leucovorin, oxaliplatin, and bevacizumab) or an equivalent thereof. The administration of these can be concurrent or sequential, as determined by the treating physician.

The anti-VEGF therapy can be a first line, second line or third line therapy. In one particular aspect, the anti-VEGF therapy is a first line therapy.

Cancer patients that are suitably treated by these methods include those suffering from at least one cancer of the type of the group: metastatic or non-metastatic rectal cancer, metastatic or non-metastatic colon cancer, metastatic or non-metastatic colorectal cancer, non-small cell lung cancer, metastatic breast cancer, non-metastatic breast cancer, renal cell carcinoma, glioblastoma multiforme, head and neck cancer, ovarian cancer, hormone-refractory prostate cancer, non-metastatic unresectable liver cancer, or metastatic or unresectable locally advanced pancreatic cancer. In one particular aspect, the cancer patient is suffering from colorectal cancer, which can be metastatic or non-metastatic.

To identify the patients suitably treated by the therapy, the genotype of a cell or tissue sample isolated from the patient is determined by assaying any suitable cell or tissue that comprises, or alternatively consists essentially of, or yet further consists of, at least one of a tumor cell, a normal cell adjacent to a tumor, a normal cell corresponding to the tumor tissue type, a blood cell, a peripheral blood lymphocyte, or combinations thereof, which can be in a form of at least one of a fixed tissue, a frozen tissue, a biopsy tissue, a resection tissue, a microdissected tissue, or combinations thereof.

Any suitable method for determining the genotype of the sample can be used in the practice of these methods. For the purpose of illustration only, such methods comprise, or alternatively consist essentially of, or yet further consist of, PCR, PCR-RFLP, sequencing, or microarray.

The methods are useful to treat patients that include but are not limited to animals, such as mammals which can include simians, ovines, bovines, murines, canines, equines, and humans.

Thus, in this aspect, the invention provides a method for treating a patient selected for an anti-VEGF therapy or identified as suitably treated by the method and in need of the therapy, the patient having a cancer. This method comprising, or alternatively consisting essentially of, or yet further consisting of,

(a) determining an intratumoral expression level of at least one gene of the group EGFR, VEGFR2 or ERCC1 in a cell or tissue sample of the corresponding cancer isolated from the patient;

(b) identifying the patient having an EGFR expression level higher than a predetermined first value; a VEGFR2 expression level higher than a predetermined second value; or an ERCC1 expression level lower than a predetermined third value; and

(c) administering to the patient identified in step (b) an effective amount of an anti-VEGF therapy, thereby treating the patient.

In another aspect, the invention is a method for treating a patient identified as suitably treated by the method and in need of the therapy, the patient having a cancer. This method comprising, or alternatively consisting essentially of, or yet further consisting of, determining an intratumoral expression level of EGFR, identifying the patient having an EGFR expression level higher than a predetermined first value, and administering to the patient an effective amount of an anti-VEGF therapy, thereby treating the patient.

In another aspect, the invention is a method for treating a patient identified as suitably treated by the method and in need of the therapy, the patient having a cancer. This method comprising, or alternatively consisting essentially of, or yet further consisting of, determining an intratumoral expression level of VEGFR2, identifying the patient having an VEGFR2 expression level higher than a predetermined second value, and administering to the patient an effective amount of an anti-VEGF therapy, thereby treating the patient.

In another aspect, the invention is a method for treating a patient identified as suitably treated by the method and in need of the therapy, the patient having a cancer. This method comprising, or alternatively consisting essentially of, or yet further consisting of, determining an intratumoral expression level of ERCC1, identifying the patient having an ERCC1 expression level lower than a predetermined third value, and administering to the patient an effective amount of an anti-VEGF therapy, thereby treating the patient.

The anti-VEGF therapies can be administered by any suitable formulation. Accordingly, a formulation comprising the necessary anti-VEGF therapy is further provided herein. The formulation can further comprise one or more preservatives or stabilizers. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1, 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, and 1.0%).

The chemotherapeutic agents or drugs can be administered as a composition. A “composition” typically intends a combination of the active agent and another carrier, e.g., compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The term carrier further includes a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Additional carriers include polymeric excipients/additives such as polyvinylpyrrolidones, ficolls (a polymeric sugar), dextrates (e.g., cyclodextrins, such as 2-hydroxypropyl-.quadrature.-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives and any of the above noted carriers with the additional provisio that they be acceptable for use in vivo. For examples of carriers, stabilizers and adjuvants, see Martin REMINGTON'S PHARM. SCI., 15th Ed. (Mack Publ. Co., Easton (1975) and Williams & Williams, (1995), and in the “PHYSICIAN'S DESK REFERENCE”, 52^{nd} ed., Medical Economics, Montvale, N.J. (1998).

Many combination chemotherapeutic regimens are known to the art, such as combinations of platinum compounds and taxanes, e.g. carboplatin/paclitaxel, capecitabine/docetaxel, the “Cooper regimen”, fluorouracil-levamisole, fluorouracil-leucovorin, fluorouracil/oxaliplatin, methotrexate-leucovorin, and the like.

Combinations of chemotherapies and molecular targeted therapies, biologic therapies, and radiation therapies are also well known to the art; including therapies such as trastuzumab plus paclitaxel, alone or in further combination with platinum compounds such as oxaliplatin, for certain breast cancers, and many other such regimens for other cancers; and the “Dublin regimen” 5-fluorouracil IV over 16 hours on days 1-5 and 75 mg/m² cisplatin IV or oxaliplatin over 8 hours on day 7, with repetition at 6 weeks, in combination with 40 Gy radiotherapy in 15 fractions over the first 3 weeks) and the “Michigan regimen” (fluorouracil plus cisplatin or oxaliplatin plus vinblastine plus radiotherapy), both for esophageal cancer, and many other such regimens for other cancers, including colorectal cancer.

In another aspect of the invention, the method for treating a patient further comprises, or alternatively consists essentially of, or yet further consists of surgical resection of a metastatic or non-metastatic solid malignant tumor and, in some aspects, in combination with radiation. Methods for treating these tumors as Stage I, Stage II, Stage III, or Stage IV by surgical resection and/or radiation are known to one skilled in the art. Guidelines describing methods for treatment by surgical resection and/or radiation can be found at the National Comprehensive Cancer Network's web site, nccn.org, last accessed on May 27, 2008.

The invention provides an article of manufacture, comprising packaging material and at least one vial comprising a solution of the chemotherapy as described herein and/or or at least one antibody or its biological equivalent with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further comprises an article of manufacture, comprising packaging material, a first vial comprising the chemotherapy and/or at least one lyophilized antibody or its biological equivalent and a second vial comprising an aqueous diluent of prescribed buffer or preservative, wherein said packaging material comprises a label that instructs a patient to reconstitute the therapeutic in the aqueous diluent to form a solution that can be held over a period of twenty-four hours or greater.

Chemotherapeutic formulations of the present invention can be prepared by a process which comprises mixing at least one antibody or biological equivalent and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing of the antibody and preservative in an aqueous diluent is carried out using conventional dissolution and mixing procedures. For example, a measured amount of at least one antibody in buffered solution is combined with the desired preservative in a buffered solution in quantities sufficient to provide the antibody and preservative at the desired concentrations. Variations of this process would be recognized by one of skill in the art, e.g., the order the components are added, whether additional additives are used, the temperature and pH at which the formulation is prepared, are all factors that can be optimized for the concentration and means of administration used.

The compositions and formulations can be provided to patients as clear solutions or as dual vials comprising a vial of lyophilized antibody that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available. Recognized devices comprising these single vial systems include those pen-injector devices for delivery of a solution such as BD Pens, BD Autojectore, Humaject® NovoPen®, B-D®Pen, AutoPen®, and OptiPen®, GenotropinPen®, Genotronorm Pen®, Humatro Pen®, Reco-Pen®, Roferon Pen®, Biojector®, Iject®, J-tip Needle-Free Injector®, Intraject®, Medi-Ject®, e.g., as made or developed by Becton Dickensen (Franklin Lakes, N.J. available at bectondickenson.com), Disetronic (Burgdorf, Switzerland, available at disetronic.com; Bioject, Portland, Oreg. (available at bioject.com); National Medical Products, Weston Medical (Peterborough, UK, available at weston-medical.com), Medi-Ject Corp (Minneapolis, Minn., available at mediject.com).

Various delivery systems are known and can be used to administer a chemotherapeutic agent of the invention, e.g., encapsulation in liposomes, microparticles, microcapsules, expression by recombinant cells, receptor-mediated endocytosis. See e.g., Wu and Wu (1987) J. Biol. Chem. 262:4429-4432 for construction of a therapeutic nucleic acid as part of a retroviral or other vector, etc. Methods of delivery include but are not limited to intra-arterial, intra-muscular, intravenous, intranasal and oral routes. In a specific embodiment, it may be desirable to administer the pharmaceutical compositions of the invention locally to the area in need of treatment; this may be achieved by, for example, and not by way of limitation, local infusion during surgery, by injection or by means of a catheter.

The agents identified herein as effective for their intended purpose can be administered to subjects or individuals identified by the methods herein as suitable for the therapy. Therapeutic amounts can be empirically determined and will vary with the pathology being treated, the subject being treated and the efficacy and toxicity of the agent.

Also provided is a therapy or a medicament comprising an effective amount of a chemotherapeutic as described herein for treatment of a human cancer patient having the polymorphism of the gene of interest as identified in the experimental examples. Further provided is a therapy comprising an anti-VEGF antibody, or alternatively an anti-VEGF therapy, for use in treating a human cancer patient having the polymorphism of the gene of interest as identified in the experimental examples.

Methods of administering pharmaceutical compositions are well known to those of ordinary skill in the art and include, but are not limited to, oral, microinjection, intravenous or parenteral administration. The compositions are intended for topical, oral, or local administration as well as intravenously, subcutaneously, or intramuscularly. Administration can be effected continuously or intermittently throughout the course of the treatment. Methods of determining the most effective means and dosage of administration are well known to those of skill in the art and will vary with the cancer being treated and the patient. and the subject being treated. Single or multiple administrations can be carried out with the dose level and pattern being selected by the treating physician.

### Kits

As set forth herein, the invention provides diagnostic methods for determining the gene expression of interest. In some embodiments, the methods use probes or primers or microarrays comprising nucleotide sequences which are complementary to the gene of interest. Accordingly, the invention provides kits for performing these methods as well as instructions for carrying out the methods of this invention. Thus, in one aspect, this invention also provides a kit for use in identifying an adjuvant cancer patient more likely to have tumor recurrence, comprising, or alternatively consisting essentially of, or yet further consisting of, suitable primers, probes and/or a microarray for determining an expression level of VEGF or VEGFR1 gene, and instructions for use therein. Examples of suitable primers and probes are provided herein.

In one aspect, the components and instructions of the kit identifies a patient as more likely to experience tumor recurrence if the VEGF gene expression level is high or higher than the predetermined first value or alternatively, when a VEGFR1 gene expression level is high or higher than the predetermined second value.

In one particular aspect, the components and instructions of the kit is used to determine if the patient is more likely to experience a shorter time to tumor recurrence than patients having the adjuvant cancer and having a VEGF gene expression level low or lower than the predetermined first value, or a VEGFR1 gene expression level low or lower than the predetermined second value.

In a further aspect, the components and instructions of the kit is used to determine if the patient as less likely to experience tumor recurrence when a VEGF gene expression level is lower than the predetermined first value, or a VEGFR1 gene expression level is low or lower than the predetermined second value.

Also provided by this invention are the components and instructions of the kit for identifying an adjuvant cancer patient more likely to experience tumor recurrence, comprising, or alternatively consisting essentially of, or yet further consisting of, determining an intratumoral expression level of VEGF gene in a cell or tissue sample of the corresponding cancer isolated from the patient, wherein a VEGF gene expression level that is high or higher than a predetermined value identifies the patient as more likely to experience tumor recurrence, or a VEGF gene expression level that is low or lower than the predetermined value identifies the patient as less likely to experience tumor recurrence.

In one aspect, the method is used to identify a patient likely to experience a shorter time to tumor recurrence than patients having the adjuvant cancer and having a VEGF gene expression level that is low or lower than the predetermined value.

Yet further provided are the components and instructions of the kit for identifying an adjuvant cancer patient more likely to experience tumor recurrence, comprising, or alternatively consisting essentially of, or yet further consisting of, determining an intratumoral expression level of VEGFR1 gene in a cell or tissue sample of the corresponding cancer isolated from the patient, wherein a VEGFR1 gene expression level that is high or higher than a predetermined value identifies the patient as more likely to experience tumor recurrence, or a VEGFR1 gene expression level that is low or lower than the predetermined value identifies the patient as less likely to experience tumor recurrence.

In one aspect, the patient is more likely to experience tumor recurrence or likely to experience a shorter time to tumor recurrence than patients having the adjuvant cancer and having a VEGFR1 gene expression level that is low or lower than the predetermined value.

Briefly and for the purpose of illustration only, one of skill in the art can determine the first and second predetermined values by comparing expression values of a gene in patients with more desirable clinical parameters to those with less desirable clinical parameters. In one aspect, a predetermined value is a gene expression value that best separates patients into a group with more desirable clinical parameter and a group with less desirable clinical parameter. Such a gene expression value can be mathematically or statistically determined with methods well known in the art.

The components and instructions of the kit are useful for the prognosis and treatment of patients suffering from at least one or more cancer of the group: metastatic or non-metastatic rectal cancer, metastatic or non-metastatic colon cancer, metastatic or non-metastatic colorectal cancer, lung cancer, head and neck cancer, non-small cell lung cancer, metastatic breast cancer, non-metastatic breast cancer, renal cell carcinoma, glioblastoma multiforme, ovarian cancer, hormone-refractory prostate cancer, non-metastatic unresectable liver cancer, or metastatic or unresectable locally advanced pancreatic cancer, prior to a surgical resection.

Suitable samples for use in the methods of this invention include, but are not limited to a fixed tissue, a frozen tissue, a biopsy tissue, a resection tissue, a microdissected tissue, or combinations thereof.

In one aspect, the kit further comprises, or alternatively consists essentially of, or yet further consists of, an anti-VEGF therapy, as defined herein, and optionally instructions for administration of the therapy. In one aspect, the amount is an effective amount to treat the cancer of the patient. In one aspect, the anti-VEGF therapy further comprises or alternatively consists essentially of, or yet further consists of, administration of a platinum drug or an equivalent thereof.

In a yet further aspect, the anti-VEGF therapy further comprises or alternatively consists essentially of, or yet further consists of, administration of a pyrimidine antimetabolite or equivalents thereof. As an example, the anti-VEGF therapy comprises or alternatively consists essentially of, or yet further consists of, administration FOLFOX/BV (5-FU, leucovorin, oxaliplatin, and bevacizumab) or XELOX/BV (capecitabine, leucovorin, oxaliplatin, and bevacizumab). The instructions can detail how to administer the therapies sequentially or concurrently.

Oligonucleotides “specific for” the gene of interest bind either to the gene of interest or bind adjacent to the gene of interest. For oligonucleotides that are to be used as primers for amplification, primers are adjacent if they are sufficiently close to be used to produce a polynucleotide comprising the gene of interest. In one embodiment, oligonucleotides are adjacent if they bind within about 1-2 kb, and preferably less than 1 kb from the gene of interest. Specific oligonucleotides are capable of hybridizing to a sequence, and under suitable conditions will not bind to a sequence differing by a single nucleotide.

The kit can comprise at least one probe and/or primer which is capable of specifically hybridizing to the gene of interest and instructions for use. The kits preferably comprise at least one of the above described nucleic acids. Preferred kits for amplifying at least a portion of the gene of interest comprise two primers, at least one of which is capable of hybridizing to the allelic variant sequence. Such kits are suitable for detection of genotype by, for example, fluorescence detection, by electrochemical detection, or by other detection.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In one embodiment, the preferred surface is silica or glass. In another embodiment, the surface is a metal electrode.

Yet other kits of the invention comprise at least one reagent necessary to perform the assay. For example, the kit can comprise an enzyme. Alternatively the kit can comprise a buffer or any other necessary reagent.

Conditions for incubating a nucleic acid probe with a test sample depend on the format employed in the assay, the detection methods used, and the type and nature of the nucleic acid probe used in the assay. One skilled in the art will recognize that any one of the commonly available hybridization, amplification or immunological assay formats can readily be adapted to employ the nucleic acid probes for use in the present invention. Examples of such assays can be found in Chard, T. (1986) AN INTRODUCTION TO RADIOIMMUNOASSAY AND RELATED TECHNIQUES Elsevier Science Publishers, Amsterdam, The Netherlands; Bullock, G. R. et al., TECHNIQUES IN IMMUNOCYTOCHEMISTRY Academic Press, Orlando, Fla. Vol. 1 (1982), Vol. 2 (1983), Vol. 3 (1985); Tijssen, P. (1985) PRACTICE AND THEORY OF IMMUNOASSAYS: LABORATORY TECHNIQUES IN BIOCHEMISTRY AND MOLECULAR BIOLOGY, Elsevier Science Publishers, Amsterdam, The Netherlands.

The test samples used in the diagnostic kits include cells, protein or membrane extracts of cells, or biological fluids such as sputum, blood, serum, plasma, or urine. The test samples may also be a tumor cell, a normal cell adjacent to a tumor, a normal cell corresponding to the tumor tissue type, blood, a peripheral blood lymphocyte, or combinations thereof. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kits can include all or some of the positive controls, negative controls, reagents, primers, sequencing markers, probes and antibodies described herein for determining the subject's genotype in the polymorphic region of the gene of interest.

As amenable, these suggested kit components may be packaged in a manner customary for use by those of skill in the art. For example, these suggested kit components may be provided in solution or as a liquid dispersion or the like.

### Other Uses for the Nucleic Acids of the Invention

The identification of the polymorphic region or the expression level of the gene of interest can also be useful for identifying an individual among other individuals from the same species. For example, DNA sequences can be used as a fingerprint for detection of different individuals within the same species. Thompson, J. S, and Thompson, eds., (1991) GENETICS IN MEDICINE, W B Saunders Co., Philadelphia, Pa. This is useful, e.g., in forensic studies.

The invention now being generally described, it will be more readily understood by reference to the following example which is included merely for purposes of illustration of certain aspects and embodiments of the present invention, and are not intended to limit the invention.

### EXPERIMENTAL DETAILS

### Example 1

Background: While wild type (wt) Kras is associated with improved outcome to anti-EGFR therapy in patients with mCRC, there are no identified predictors of outcome for FOLFOX/BV. Kras status was evaluated together with expression of genes involved in angiogenesis, DNA repair and 5-FU metabolism in 68 patients treated with FOLFOX/BV or XELOX/BV. These genes included VEGF, VEGF-receptor 2 (KDR), Cox-2, IL 6 and 8, chemokine-receptors 1 & 2, EGFR and ERCC-1.

Methods: Tissue samples from 68 patients with mCRC were analyzed. mRNA was extracted from laser-capture-microdissected tumor tissue. cDNA was prepared by reverse transcription and quantitation of the candidate genes was performed using a fluorescence-based real-time detection method (TaqMan®). Allele specific RT-PCR was performed to determine Kras mutation status in codons 12 and 13. Primers and probes used are included in Table 1.

TABLE 1
Primers used in real-time PCR

| Gene | Forward Primer (5′-3′) | Reverse Primer (5′-3′) | Taqman Probe (5′-3′) |
|---|---|---|---|
| β-actin | GAGCGCGGCTACAGC | TCCTTAATGTCACGCAC | ACCACCACGGCCGAGCG |
| | TT (SEQ ID NO. 1) | GATTT (SEQ ID NO. 2) | G (SEQ ID NO. 3) |
| EGFR | TGCGTCTCTTGCCGGA | GGCTCACCCTCCAGAA | ACGCATTCCCTGCCTCGG |
| | AT (SEQ ID NO. 4) | GCTT (SEQ ID NO. 5) | CTG (SEQ ID NO. 6) |
| VEGFR2 | CCTGTGGCTCTGCGTG | CTGAGCCTGGGCAGAT | CACTAGGCAAACCCACA |
| | GA (SEQ ID NO. 7) | CAAG (SEQ ID NO. 8) | GAGGCGGC (SEQ ID NO. 9) |
| ERCC1 | GGGAATTTGGCGACG | GCGGAGGCTGAGGAAC | CACAGGTGCTCTGGCCCA |
| | TAATTC (SEQ ID NO. 10) | AG (SEQ ID NO. 11) | GCACATA (SEQ ID NO. 12) |

Results: There were 68 patients (38 males, 30 females), median age: 56 years (range 29-81). All received first line 5-FU, oxaliplatin and BV (28 FOLFOX/BV, 40 XELOX/BV). Radiologic response: 1 CR, 39/68 (57%) PR, 27/68 (40%) SD, and 1 PD. Median OS is not reached. At a median follow-up of 32.0 months (mo) (range: 2.3-47.8 mo), the median PFS was 12.4 mo (95% CI: 9.8-15.2). Kras mutation was identified in 39 patients (57%). RR was 64% in patients with wt Kras and 52% in patients with mutant Kras (p=0.33). PFS was significantly longer for patients with wt kras compared to patients with mutant kras (13.7 mo [95% CI: 6.9-13.2] versus 8.3 mo [95% CI: 6.9-13.2], P=0.039). High EGFR (median PFS: 15.2 mo; 95% CI 11.7-16.5 mo), high VEGFR2 (median PFS: 13.9 mo; 95% CI 11.0-16.5 mo), and low ERCC1 (median PFS: 12.4 mo; 95% CI 10.9-16.4 mo) were associated with longer PFS compared to low EGFR (median PFS: 7.9 mo; 95% CI 6.9-11.0 mo, P=0.040), low VEGFR2 (median PFS: 7.2 mo; 95% CI 6.5-8.1 mo, P=0.032), and high ERCC1 (median PFS: 9.6 mo; 95% CI 5.8-15.2 mo, P=0.045).

Conclusions: To our knowledge, this is the first report of a potential association between Kras status as well as gene expression levels of VEGFR2, ERCC-1 and EGFR and clinical outcome to FOLFOX/BV therapy in patients with mCRC.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. A method for identifying a cancer patient suitable for an anti-VEGF therapy comprising determining an intratumoral expression level of at least one gene of the group EGFR, VEGFR2 or ERCC1 in a cell or tissue sample of the corresponding cancer isolated from the patient, wherein the presence of:
(a) an EGFR expression level higher than a predetermined first value;
(b) a VEGFR2 expression level higher than a predetermined second value; or
(c) an ERCC1 expression level lower than a predetermined third value,
identifies the patient as suitable for the therapy, or the presence of none of (a) to (c) identifies the patient as not suitable for the therapy.

2. The method of claim 1, wherein the presence of:
(a) an EGFR expression level higher than a predetermined first value;
(b) a VEGFR2 expression level higher than a predetermined second value; or
(c) an ERCC1 expression level lower than a predetermined third value,
identifies the patient as suitable for the therapy.

3. The method of claim 1, wherein the presence of none of (a) to (c) identifies the patient as not suitable for the therapy.

4. A method for identifying a cancer patient suitable for an anti-VEGF therapy comprising determining an intratumoral expression level of EGFR in a cell or tissue sample of the corresponding cancer isolated from the patient, wherein an EGFR expression level higher than a predetermined value identifies the patient as suitable for the therapy, or an EGFR expression level lower than the predetermined value identifies the patient as not suitable for the therapy.

5. The method of claim 4, wherein a cancer patient suitable for the anti-VEGF therapy is a cancer patient having a longer progress free survival than a patient having an EGFR expression level lower than the predetermined value and having the cancer and receiving the anti-VEGF therapy.

6. A method for identifying a cancer patient suitable for an anti-VEGF therapy comprising determining an intratumoral expression level of VEGFR1 in a cell or tissue sample of the corresponding cancer isolated from the patient, wherein a VEGFR1 expression level higher than a predetermined value identifies the patient as suitable for the therapy, or a VEGFR1 expression level lower than the predetermined value identifies the patient as not suitable for the therapy.

7. The method of claim 6, wherein a cancer patient suitable for the anti-VEGF therapy is a cancer patient having a longer progress free survival than a patient having an VEGFR1 expression level lower than the predetermined value and having the cancer and receiving the anti-VEGF therapy.

8. A method for identifying a cancer patient suitable for an anti-VEGF therapy comprising determining an intratumoral expression level of ERCC1 in a cell or tissue sample of the corresponding cancer isolated from the patient, wherein an ERCC1 expression level lower than a predetermined value identifies the patient as suitable for the therapy, or an ERCC1 expression level higher than the predetermined value identifies the patient as not suitable for the therapy.

9. The method of claim 4, wherein a cancer patient suitable for the anti-VEGF therapy is a cancer patient having a longer progress free survival than a patient having an ERCC1 expression level higher than the predetermined value and having the cancer and receiving the anti-VEGF therapy.

10. A method selecting a cancer patient for an anti-VEGF therapy comprising determining an intratumoral expression level of at least one gene of the group EGFR, VEGFR2 or ERCC1 in a cell or tissue sample of the corresponding cancer isolated from the patient, wherein the patient is selected if one or more of:
(a) an EGFR expression level higher than a predetermined first value;
(b) a VEGFR2 expression level higher than a predetermined second value; or
(c) an ERCC1 expression level lower than a predetermined third value,
is present, or the patient is not selected if none of (a) to (c) is present.

11. The method of claim 10, wherein the patient is selected if one or more of:
(a) an EGFR expression level higher than a predetermined first value;
(b) a VEGFR2 expression level higher than a predetermined second value; or
(c) an ERCC1 expression level lower than a predetermined third value,
is present.

12. The method of claim 10, wherein the patient is not selected if none of (a) to (c) is present.

13. The method of claim 1, wherein the anti-VEGF therapy comprises administration of an anti-VEGF antibody or an equivalent thereof.

14. The method of claim 13, wherein the therapy further comprises administration of a platinum drug or an equivalent thereof.

15. The method of claim 13 or 14 wherein the therapy further comprises administration of a pyrimidine antimetabolite or equivalents thereof.

16. The method of claim 1, wherein the anti-VEGF therapy comprises administration of FOLFOX/BV (5-FU, leucovorin, oxaliplatin, and bevacizumab) or an equivalent thereof or XELOX/BV (capecitabine, leucovorin, oxaliplatin, and bevacizumab) or an equivalent thereof.

17. The method of claim 14, wherein the administration of the anti-VEGF antibody or an equivalent thereof, and a platinum drug or an equivalent thereof, and/or a pyrimidine antimetabolite drug or equivalents thereof is concurrent or sequential.

18. The method of claim 1, wherein the anti-VEGF therapy is a first line therapy.

19. The method of claim 1 wherein the cancer patient is suffering from at least one cancer of the type of the group metastatic or non-metastatic rectal cancer, metastatic or non-metastatic colon cancer, metastatic or non-metastatic colorectal cancer, non-small cell lung cancer, metastatic breast cancer, non-metastatic breast cancer, renal cell carcinoma, glioblastoma multiforme, ovarian cancer, hormone-refractory prostate cancer, non-metastatic unresectable liver cancer, or metastatic or unresectable locally advanced pancreatic cancer.

20. The method of claim 1, wherein the cancer patient is suffering from colorectal cancer.

21. The method of claim 1, wherein the cancer patient is suffering from metastatic colorectal cancer.

22. The method of claim 1, wherein the gene expression level is determined by a method that comprises determining the amount of a mRNA transcribed from the gene.

23. The method of claim 1, wherein the gene expression level is determined by a method comprising one or more of in situ hybridization, PCR, real-time PCR, or microarray.

24. The method of claim 1, wherein the sample is at least one of a fixed tissue, a frozen tissue, a biopsy tissue, a resection tissue, a microdissected tissue, or combinations thereof.

25. The method of claim 1, wherein the patient is a human patient.

26. A method for treating a cancer patient, comprising administering an anti-VEGF therapy to a cancer patient selected for the therapy based on one or more of:
(a) an EGFR expression level higher than a predetermined first value,
(b) a VEGFR2 expression level higher than a predetermined second value, or
(c) an ERCC1 expression level lower than a predetermined third value,
in a sample isolated from the patient, thereby treating the patient.

27. The method of claim 26, wherein the patient was selected by a method comprising determining an intratumoral expression level of at least one gene of the group EGFR, VEGFR2 or ERCC1 in a cell or tissue sample of the corresponding cancer isolated from the patient.

28. The method of claim 26, wherein the anti-VEGF therapy comprises administration of anti-VEGF antibody or an equivalent thereof.

29. The method of claim 28, wherein the therapy further comprises administration of a platinum drug or an equivalent thereof.

30. The method of claim 28 or 29 wherein the therapy further comprises administration of a pyrmidine antimetabolite or equivalents thereof.

31. The method of claim 26, wherein the anti-VEGF therapy comprises administration of FOLFOX/BV (5-FU, leucovorin, oxaliplatin, and bevacizumab) or an equivalent thereof or XELOX/BV (capecitabine, leucovorin, oxaliplatin, and bevacizumab) or an equivalent thereof.

32. The method of claim 28, wherein the administration of an anti-VEGF antibody or an equivalent thereof, and a platinum drug or an equivalent thereof, and/or a pyrmidine antimetabolite or equivalents thereof is concurrent or sequential.

33. The method of claim 28, wherein the cancer patient is suffering from at least one cancer of the type of the group metastatic or non-metastatic rectal cancer, metastatic or non-metastatic colon cancer, metastatic or non-metastatic colorectal cancer, non-small cell lung cancer, metastatic breast cancer, non-metastatic breast cancer, renal cell carcinoma, glioblastoma multiforme, ovarian cancer, hormone-refractory prostate cancer, non-metastatic unresectable liver cancer, or metastatic or unresectable locally advanced pancreatic cancer.

34. The method of claim 28, wherein the cancer patient is suffering from colorectal cancer.

35. The method of claim 28, wherein the cancer patient is suffering from metastatic colorectal cancer.

36. The method of claim 28, wherein the sample is at least one of a fixed tissue, a frozen tissue, a biopsy tissue, a resection tissue, a microdissected tissue, or combinations thereof.

37. Use of an anti-VEGF therapy for the therapy of a cancer patient identified for suitable for the therapy based on the methods of claim 1.

38.-47. (canceled)

48. A panel of probes and/or primers and/or a microarray to determine an intratumoral expression level of at least two genes of the group EGFR, VEGFR2 or ERCC1 in a cell or tissue sample.
